(19) United States
(12) Patent Application Publication
(10) Pub. No.: US 2024/0215956 A1
(43) Pub. Date: Jul. 4, 2024

(54) SYSTEM FOR ADJUSTING ORIENTATION OF 4D ULTRASOUND IMAGE

(71) Applicant: GE Precision Healthcare LLC, Waukesha, WI (US)

(72) Inventors: Zhiqiang Jiang, Wuxi (CN); Yao Ding, Wuxi (CN); Yan Wei, Wuxi (CN)

(21) Appl. No.: 18/538,256

(22) Filed: Dec. 13, 2023

(30) Foreign Application Priority Data
Dec. 30, 2022 (CN) 202211729845.5

(51) Int. Cl. A61B 8/08; A61B 8/00; G06T 3/40

(52) U.S. Cl. CPC A61B 8/5223; A61B 8/085; A61B 8/463; A61B 8/5207; G06T 3/40; A61B 8/0866

(57) ABSTRACT

A method for adjusting an orientation of a 4D ultrasound image includes: acquiring 4D ultrasound data about a tissue to be imaged; processing the 4D ultrasound data to generate a 4D ultrasound image, the 4D ultrasound image including a plurality of image frames; identifying at least one anatomical feature of interest and a current orientation thereof; and adjusting the 4D ultrasound image, such that the at least one anatomical feature of interest is always maintained at a target orientation in the plurality of image frames. Further provided in the present application are a system for adjusting an orientation of a 4D ultrasound image and a non-transitory computer-readable medium.

### CROSS-REFERENCE TO RELATED APPLICATION

The present application claims priority to Chinese Patent Application No. 202211729845.5, filed on Dec. 30, 2022. The entire contents of the above-listed application are incorporated by reference herein in their entirety.

### TECHNICAL FIELD

The present invention relates to the field of medical imaging, and relates in particular to a method for adjusting an orientation of a 4D ultrasound image, a system for adjusting an orientation of a 4D ultrasound image, and a non-transitory computer-readable medium.

### BACKGROUND

Ultrasound imaging technology generally uses a probe to send an ultrasonic signal to a part to be scanned and receive an ultrasonic echo signal. The echo signal is further processed to obtain an ultrasound image of the part to be scanned. Based on this principle, ultrasound imaging is suitable for real-time and non-destructive scanning of subjects to be scanned.

The four-dimensional (4D) ultrasound imaging technique is one type of the above technology, and enables continuous three-dimensional volume imaging of a tissue to be imaged in the time dimension, thereby providing a physician with more abundant information. 4D imaging is widely applied to examination of a tissue to be imaged, such as a fetus, the heart, etc. In the 4D imaging process, when images of an anatomical feature of interest are not acquired, it is generally necessary to move the probe, or to adjust parameters such as an orientation of a 4D ultrasound image by using an adjustment function of an ultrasound machine, until a satisfactory image appears. 4D ultrasound changes dynamically over time, which further increases the difficulty of the above adjustment. Furthermore, when an object to be scanned is mobile, the above adjustment will become even more difficult.

### SUMMARY

The aforementioned defects, deficiencies, and problems are solved herein, and these problems and solutions will be understood through reading and understanding the following description.

Provided in some embodiments of the present application is a method for adjusting an orientation of a 4D ultrasound image, comprising: acquiring 4D ultrasound data about a tissue to be imaged; processing the 4D ultrasound data to generate a 4D ultrasound image, the 4D ultrasound image comprising a plurality of image frames; identifying at least one anatomical feature of interest and a current orientation thereof; and adjusting the 4D ultrasound image, such that the at least one anatomical feature of interest is always maintained at a target orientation in the plurality of image frames.

Provided in some embodiments of the present application is a system for adjusting an orientation of a 4D ultrasound image, comprising: a probe, configured to receive 4D ultrasound data about a tissue to be imaged; a processor, configured to perform the following method: acquiring 4D ultrasound data about a tissue to be imaged, processing the 4D ultrasound data to generate a 4D ultrasound image, the 4D ultrasound image comprising a plurality of image frames, identifying at least one anatomical feature of interest and an orientation thereof, and adjusting the 4D ultrasound image, such that the at least one anatomical feature of interest is always maintained at a target orientation in the plurality of image frames; and a display, receiving a signal from the processor and performing a display operation.

Provided in some embodiments of the present application is a non-transitory computer-readable medium, storing a computer program, the computer program having at least one code segment, and the at least one code segment being executable by a machine to cause the machine to perform the following method: acquiring 4D ultrasound data about a tissue to be imaged; processing the 4D ultrasound data to generate a 4D ultrasound image, the 4D ultrasound image comprising a plurality of image frames; identifying at least one anatomical feature of interest and a current orientation thereof; and adjusting the 4D ultrasound image, such that the at least one anatomical feature of interest is always maintained at a target orientation in the plurality of image frames.

It should be understood that the brief description above is provided to introduce, in a simplified form, concepts that will be further described in the detailed description. The brief description above is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### BRIEF DESCRIPTION OF THE DRAWINGS

The present application will be better understood by reading the following description of non-limiting embodiments with reference to the accompanying drawings, wherein:

FIG. 1 is a schematic diagram of an ultrasound imaging system according to some embodiments of the present application;

FIG. 2 is a flowchart of an ultrasound imaging method in some embodiments of the present application;

FIG. 3 is a schematic diagram showing how adjustments are made to a volumetric image in some embodiments of the present application;

FIG. 4 is a schematic diagram showing how orientation adjustments are made in a plurality of volumetric images in some embodiments of the present application;

FIG. 5 is a schematic diagram of a neural network having one or more nodes/neurons in some embodiments of the present application;

FIG. 6 is a schematic diagram of input and output connections of a neuron in some embodiments of the present application;

FIG. 7 is a flowchart showing how a 4D ultrasound image including a plurality of anatomical features of interest is adjusted in some embodiments of the present application;

FIG. 8 is a schematic diagram of simultaneous display of a plurality of adjusted 4D ultrasound images in some embodiments of the present application; and

FIG. 9 is a schematic diagram of a 4D ultrasound image displayed in an enlarged manner in some embodiments of the present application.

### DETAILED DESCRIPTION

Specific implementations of the present invention will be described in the following. It should be noted that in the specific description of the implementations, it is impossible to describe all features of the actual implementations of the present invention in detail, for the sake of brief description. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for a person of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless otherwise defined, the technical or scientific terms used in the claims and the description should be as they are usually understood by those possessing ordinary skill in the technical field to which they belong. “First”, “second”, and similar words used in the present invention and the claims do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

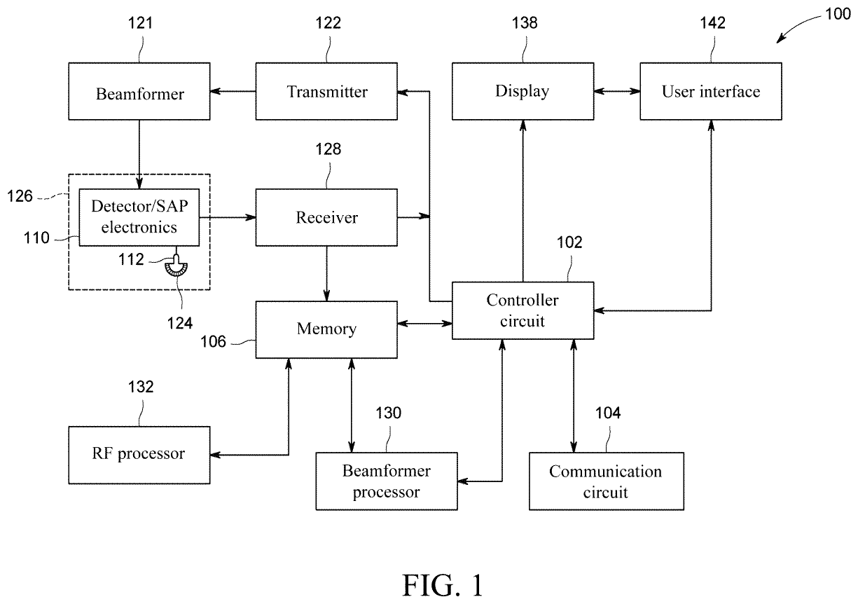

FIG. 1 shows a schematic block diagram of an embodiment of an ultrasound imaging system 100. The ultrasound imaging system 100 may include a controller circuit 102, a display 138, a user interface 142, a probe 126, and a memory 106, which can be operatively connected to a communication circuit 104.

The controller circuit 102 is configured to control operation of the ultrasound imaging system 100. The controller circuit 102 may include one or more processors. Optionally, the controller circuit 102 may include a central processing unit (CPU), one or more microprocessors, a graphics processing unit (GPU), or any other electronic assembly capable of processing inputted data according to a specific logic instruction. Optionally, the controller circuit 102 may include and/or represent one or more hardware circuits or circuitry, the hardware circuits or circuitry including, connecting, or including and connecting one or more processors, controllers, and/or other hardware logic-based devices. Additionally or alternatively, the controller circuit 102 may execute an instruction stored on a tangible and non-transitory computer-readable medium (e.g., the memory 106).

The controller circuit 102 may be operatively connected to and/or control the communication circuit 104. The communication circuit 104 is configured to receive and/or transmit information along a bidirectional communication link with one or more optional ultrasound imaging systems, remote servers, etc. The remote server may represent and include patient information, a machine learning algorithm, a remotely stored medical image from a previous scan and/or diagnosis and treatment period of a patient, etc. The communication circuit 104 may represent hardware for transmitting and/or receiving data along a bidirectional communication link. The communication circuit 104 may include a transceiver, a receiver, a transceiver, etc., and associated circuitry (e.g., an antenna) for communicating (e.g., transmitting and/or receiving) in a wired and/or wireless manner with the one or more optional ultrasound imaging systems, remote servers, etc. For example, protocol firmware for transmitting and/or receiving data along a bidirectional communication link may be stored in the memory 106 accessed by the controller circuit 102. The protocol firmware provides network protocol syntax to the controller circuit 102 so as to assemble a data packet, establish and/or segment data received along the bidirectional communication link, and so on.

The bidirectional communication link may be a wired (e.g., by means of a physical conductor) and/or wireless communication (e.g., utilizing a radio frequency (RF)) link for exchanging data (e.g., a data packet) between the one or more optional ultrasound imaging systems, remote servers, etc. The bidirectional communication link may be based on a standard communication protocol, such as Ethernet, TCP/IP, WiFi, 802.11, a customized communication protocol, Bluetooth, etc.

The controller circuit 102 is operatively connected to the display 138 and the user interface 142. The display 138 may include one or more liquid crystal displays (e.g., light emitting diode (LED) backlights), organic light emitting diode (OLED) displays, plasma displays, CRT displays, and the like. The display 138 may display patient information, one or more medical images and/or videos, a graphical user interface, or an assembly received by the display 138 from the controller circuit 102, one or more 2D, 3D, or 4D ultrasound image data sets from ultrasound data stored in the memory 106, or anatomical measurement, diagnosis, processing information, etc., currently acquired in real time.

The user interface 142 controls the operation of the controller circuit 102 and the ultrasound imaging system 100. The user interface 142 is configured to receive an input from a clinician and/or an operator of the ultrasound imaging system 100. The user interface 142 may include a keyboard, a mouse, a touch pad, one or more physical buttons, and the like. Optionally, the display 138 may be a touch screen display that includes at least a portion of the user interface 142. For example, a portion of the user interface 142 may correspond to a graphical user interface (GUI) that is generated by the controller circuit 102 and that is shown on the display 138. The touch screen display may detect the presence of a touch from the operator on the display 138, and may also identify the position of the touch relative to the surface area of the display 138. For example, a user may select, by touching or contacting the display 138, one or more user interface assemblies of the user interface (GUI) shown on the display. User interface assemblies may correspond to icons, text boxes, menu bars, etc., shown on the display 138. A clinician may select, control, and use a user interface assembly, interact with same, and so on, so as to send an instruction to the controller circuit 102 to perform one or more operations described in the present application. For example, touch can be applied using at least one among a hand, a glove, a stylus, and the like.

The memory 106 includes a parameter, an algorithm, one or more ultrasound examination protocols, a data value, and the like used by the controller circuit 102 to perform one or more operations described in the present application. The memory 106 may be a tangible and non-transitory computer-readable medium such as a flash memory, a RAM, a ROM, an EEPROM, etc. The memory 106 may include a set of learning algorithms (e.g., a convolutional neural network algorithm, a deep learning algorithm, a decision tree learning algorithm, etc.) configured to define an image analysis algorithm. During execution of the image analysis algorithm, the controller circuit 102 is configured to identify an anatomical feature of interest. Optionally, an image analysis algorithm may be received by means of the communication circuit 104 along one among bidirectional communication links, and stored in the memory 106. The means of identifying an anatomical feature of interest will be described in detail below.

With continued reference to FIG. 1, the ultrasound imaging system 100 may include the probe 126, the probe 126 having a transmitter 122, a transmission beamformer 121, and a detector/SAP electronics 110. The detector/SAP electronics 110 may be used to control switching of transducer elements 124. The detector/SAP electronics 110 may also be used to group the transducer elements 124 into one or more sub-holes.

The probe 126 may be configured to acquire ultrasound data or information from tissues to be imaged (e.g., a fetus, organs, blood vessels, heart, bones, etc.). The probe 126 is communicatively connected to the controller circuit 102 by means of the transmitter 122. The transmitter 122 transmits a signal to the transmission beamformer 121 on the basis of acquisition settings received by the controller circuit 102. The acquisition settings may define the amplitude, pulse width, frequency, gain setting, scanning angle, power, time gain compensation (TGC), resolution, and the like of ultrasonic pulses emitted by the transducer elements 124. The transducer elements 124 emit a pulsed ultrasonic signal into a patient (e.g., the body). The acquisition settings may be defined by a user operating the user interface 142. The signal transmitted by the transmitter 122, in turn, drives a plurality of transducer elements 124 within a transducer array 112.

The transducer elements 124 transmit a pulsed ultrasonic signal to a body (e.g., a patient) or a volume that corresponds to an acquisition setting along one or more scanning planes. The ultrasonic signal may include, for example, one or more reference pulses, one or more push pulses (e.g., shear waves), and/or one or more pulsed wave Doppler pulses. At least a portion of the pulsed ultrasonic signal is backscattered from the tissue to be imaged (e.g., the organ, bone, heart, breast tissue, liver tissue, cardiac tissue, prostate tissue, newborn brain, embryo, abdomen, etc.) to produce an echo. Depending on the depth or movement, the echo is delayed in time and/or frequency, and received by the transducer elements 124 within the transducer array 112. The ultrasonic signal may be used for imaging, for producing and/or tracking the shear wave, for measuring changes in position or velocity within the anatomical structure and compressive displacement difference (e.g., strain) of the tissue, and/or for treatment and other applications. For example, the probe 126 may deliver low energy pulses during imaging and tracking, deliver medium and high energy pulses to produce shear waves, and deliver high energy pulses during treatment.

The transducer elements 124 convert a received echo signal into an electrical signal that can be received by a receiver 128. The receiver 128 may include one or more amplifiers, analog/digital converters (ADCs), and the like. The receiver 128 may be configured to amplify the received echo signal after appropriate gain compensation, and convert these analog signals received from each transducer element 124 into a digitized signal that is temporally uniformly sampled. The digitized signals representing the received echoes are temporarily stored in the memory 106. The digitized signals correspond to backscattered waves received by each transducer element 124 at different times. After being digitized, the signal may still retain the amplitude, frequency, and phase information of the backscattered wave.

Optionally, the controller circuit 102 may retrieve a digitized signal stored in the memory 106 for use in a beamformer processor 130. For example, the controller circuit 102 may convert the digitized signal into a baseband signal or compress the digitized signal.

The beamformer processor 130 may include one or more processors. If desired, the beamformer processor 130 may include a central processing unit (CPU), one or more microprocessors, or any other electronic assembly capable of processing inputted data according to specific logic instructions. Additionally or alternatively, the beamformer processor 130 may execute instructions stored on a tangible and non-transitory computer-readable medium (e.g., the memory 106) to perform beamforming computation using any suitable beamforming method, such as adaptive beamforming, synthetic emission focusing, aberration correction, synthetic aperture, clutter suppression, and/or adaptive noise control, among others. If desired, the beamformer processor 130 may be integrated with and/or be part of the controller circuit 102. For example, operations described as being performed by the beamformer processor 130 may be configured to be performed by the controller circuit 102.

The beamformer processor 130 performs beamforming on the digitized signal of the transducer elements, and outputs a radio frequency (RF) signal. The RF signal is then provided to an RF processor 132 for processing the RF signal. The RF processor 132 may include one or more processors. If desired, the RF processor 132 may include a central processing unit (CPU), one or more microprocessors, or any other electronic assembly capable of processing inputted data according to specific logic instructions. Additionally or alternatively, the RF processor 132 may execute instructions stored on a tangible and non-transitory computer-readable medium (e.g., the memory 106). If desired, the RF processor 132 may be integrated with and/or be part of the controller circuit 102. For example, operations described as being performed by the RF processor 132 may be configured to be performed by the controller circuit 102.

The RF processor 132 may generate, for a plurality of scanning planes or different scanning modes, different ultrasound image data types and/or modes, e.g., B-mode, color Doppler (e.g., color blood flow, velocity/power/variance), tissue Doppler (velocity), and Doppler energy, on the basis of a predetermined setting of a first model. For example, the RF processor 132 may generate tissue Doppler data for multiple scanning planes. The RF processor 132 acquires information (e.g., I/Q, B-mode, color Doppler, tissue Doppler, and Doppler energy information) related to multiple data pieces, and stores data information in the memory 106. The data information may include time stamp and orientation/rotation information.

Optionally, the RF processor 132 may include a composite demodulator (not shown) for demodulating an RF signal to generate an IQ data pair representing an echo signal. The RF or IQ signal data may then be provided directly to the memory 106 so as to be stored (e.g., stored temporarily). As desired, output of the beamformer processor 130 may be delivered directly to the controller circuit 102.

The controller circuit 102 may be configured to process acquired ultrasound data (e.g., RF signal data or an IQ data pair), and prepare and/or generate an ultrasound image data frame representing the anatomical structure of interest so as to display same on the display 138. The acquired ultrasound data may be processed by the controller circuit 102 in real time when an echo signal is received in a scanning or treatment process of ultrasound examination. Additionally or alternatively, the ultrasound data may be temporarily stored in the memory 106 in a scanning process, and processed in a less real-time manner in live or offline operations.

The memory 106 may be used to store processed frames of acquired ultrasound data that are not scheduled to be immediately displayed, or may be used to store post-processed images (e.g., shear wave images and strain images), firmware or software corresponding to, for example, a graphical user interface, one or more default image display settings, programmed instructions, and the like. The memory 106 may store medical images, such as a 4D ultrasound image data set of ultrasound data, wherein such a 4D ultrasound image data set is accessed to present real-time 3D images. For example, a 4D ultrasound image data set may be mapped to the corresponding memory 106 and one or more reference planes. Processing of ultrasound data that includes the ultrasound image data set may be based in part on user input, e.g., a user selection received at the user interface 142.

During actual use of 4D imaging by a user, it is often necessary to adjust the imaging. One reason is that volumetric data of an anatomical feature of interest that needs to be imaged has not been acquired, and at this time, the ultrasound probe needs to be moved until an ultrasonic echo signal from the location of the anatomical feature of interest can be received. Another reason is that even if the volumetric data of the anatomical feature of interest can be acquired, the positioning of the rendered 4D ultrasound image may be unsatisfactory, for example, the anatomical feature of interest in the 4D ultrasound image may be oriented towards the back of the image and thus obscured. Furthermore, if the tissue to be imaged is movable or the probe is spatially displaced, then in the 4D ultrasound imaging process, the location of the anatomical feature of interest may vary over time.

A more detailed description is made using 4D imaging of a fetus as an example. When 4D imaging needs to be performed on a certain organ of a fetus (e.g., fingers of the fetus as an anatomical feature of interest), if said organ does not appear in the field of view of a 4D ultrasound image (e.g., is obscured by the head of the fetus), the scanning operator needs to move the ultrasound probe or operate a relevant function button on the ultrasound imaging system to adjust the orientation of the current 4D ultrasound image, until the fingers are exposed in the field of view. However, on one hand, this process is quite time consuming, and on the other hand, adjusting volumetric images is very challenging to inexperienced users. In addition, even if an image of the fingers of the fetus is successfully acquired in the current frame, the fingers of the fetus are likely to be obscured again quickly during movement due to the irregular activity of the fetus.

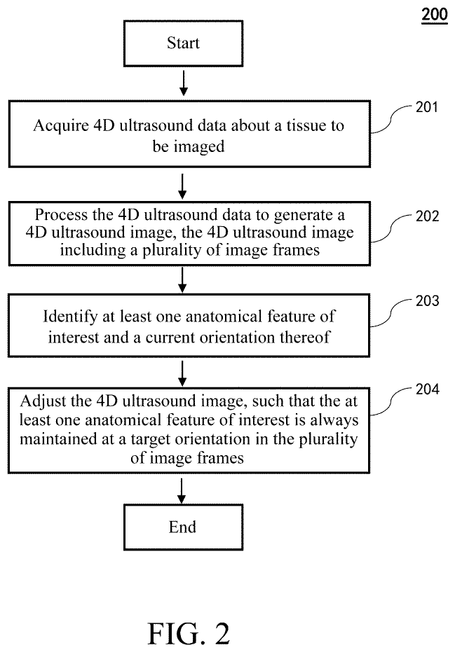

In view of this, improvements are provided in the embodiments of the present application. With reference to FIG. 2, embodiments of the present application are described in detail. FIG. 2 shows a flowchart 200 showing how an orientation of a 4D ultrasound image is adjusted in some embodiments of the present application.

In step 201, 4D ultrasound data about a tissue to be imaged is acquired. The process may be implemented by the processor of the controller circuit 102 described above. The tissue to be imaged may differ depending on the actual scan requirements, and may be any one of a fetus, a body organ, and a lesion. In one example, the tissue may be a fetus, and correspondingly, the 4D ultrasound data is an ultrasonic echo signal about the fetus. The 4D ultrasound data may be understood to be real-time volumetric ultrasound data, that is, volumetric ultrasound data recorded in the time dimension.

Furthermore, a variety of acquisition approaches may be used. In one example, the 4D ultrasound data comes from a real-time ultrasonic scan. Using fetal examination as an example, the 4D ultrasound data may come from a probe being used for fetal examination (e.g., a 4D probe). The probe receives the 4D ultrasound data from the tissue to be imaged, i.e., the fetus, and sends the data to the processor for further processing. At such time, the 4D ultrasound data may be understood as being acquired from the tissue to be imaged in real time. In another example, the 4D ultrasound data comes from data in a memory. There may be a variety of types of memory. For example, the memory may be the memory 106 of the ultrasound imaging system described above. Correspondingly, the 4D ultrasound data may be 4D ultrasound data already stored in the ultrasound imaging system. Such ultrasound data may come from data saved in a previous scan. Furthermore, the 4D ultrasound data may also be 4D ultrasound data stored in a remote server, e.g., a cloud server.

In step 202, the 4D ultrasound data is processed to generate a 4D ultrasound image, the 4D ultrasound image including a plurality of image frames. The process may be implemented by the processor. Each image frame may be understood to be a 3D render or a 3D volumetric image. A plurality of continuous 3D volumetric images are combined to constitute a 4D ultrasound image. In one example, a 4D render may be generated by means of a ray projection technique, such that the fetus may be depicted from the perspective of the ultrasound probe using the volumetric ultrasound data. For example, the 4D render may depict a volume (e.g., from volumetric ultrasound data) corresponding to the external physical appearance of the fetus. Furthermore, the 4D render may further undergo shading to present a better sense of depth to the user. A variety of shading methods may be used. For example, a plurality of surfaces may be defined based on the volumetric ultrasound data, and/or voxel data may undergo shading via ray projection. According to one embodiment, a gradient may be calculated at each pixel. The processor may calculate an amount of light corresponding to the location of each pixel, and apply one or more shading methods on the basis of the gradient and the particular light direction. When a 4D render is generated, the processor may further use a plurality of light sources as inputs. In an example, when performing the ray projection, the processor may calculate how much light is reflected, scattered, or transmitted from each voxel in a particular view direction along each ray. This may involve summing contributions from the plurality of light sources (e.g., point light sources). The processor may calculate contributions from all voxels in the volume. The processor may then synthesize values from all voxels, or interpolated values from adjacent voxels, in order to calculate final values of pixels displayed on the 4D render. While the foregoing example describes an embodiment in which the voxel values are integrated along the rays, the 4D render may also be calculated according to other techniques, such as by using the highest value along each ray, by using the average value along each ray, or by using any other volume rendering technique, and examples are not exhaustively enumerated herein.

In step 203, at least one anatomical feature of interest and a current orientation thereof are identified. The process may be implemented by the processor. An arbitrary method may be used for identifying the anatomical feature of interest and the orientation thereof. For example, the image analysis algorithm described above may be used. An exemplary description is provided below.

In one example, the identified object may be a 4D ultrasound image obtained by processing the 4D ultrasound data. Alternatively, the object may be each frame of volumetric ultrasound images in a 4D ultrasound image sequence. Correspondingly, an identified target is an external physical feature in these image frames. Using the fetus as an example, the identified target may be an external physical feature such as a limb. In some examples, the one or more anatomical features may include one or more facial features, such as the nose, the mouth, one or both eyes, one or both cars, and the like. In some examples, a facial recognition algorithm may be employed to perform a search and to then automatically identify the one or more facial features.

However, in a preferred embodiment, the step of identifying at least one anatomical feature of interest includes identifying the at least one anatomical feature of interest in the 4D ultrasound data. That is, the identified object is not the 4D ultrasound image, but the 4D ultrasound data. Unprocessed 4D ultrasound data will retain more abundant imaging information, and accordingly is capable of providing more comprehensive identification results. By way of example, if an external physical feature in the 4D ultrasound image is identified, then the anatomical structure oriented towards the back of the current 4D ultrasound image cannot be identified as it is not present in the ultrasound image. Alternatively, if some anatomical structures are obscured by other anatomical structures, the structures also cannot be identified as they are not present in the ultrasound image. However, if the 4D ultrasound data is used as the identified object, the above-described situations will not occur. The processor can perform identification according to the overall 4D ultrasound data, so as to determine all anatomical features of interest present therein and to determine orientations thereof.

In step 204, the 4D ultrasound image is adjusted such that the at least one anatomical feature of interest is always maintained at a target orientation in the plurality of image frames. The process may be implemented by the processor. The target orientation may be set by the ultrasound imaging system in advance, or may be customized by the user. The definition criteria for the target orientation may differ according to different anatomical features of interest, for example, the target orientation may be a standard orientation of the anatomical feature of interest. Alternatively, the target orientation may be an orientation that facilitates observation of the anatomical feature of interest by an ultrasound scanning physician. After knowing the current orientation of the at least one anatomical feature of interest in the current 4D ultrasound data or 4D ultrasound image, and after determining the target orientation thereof, the processor may adjust the 4D ultrasound image according to the difference between the current orientation and the target orientation. The adjustment may be performed in a three-dimensional coordinate system to ensure that the orientation of the at least one anatomical feature of interest satisfies the requirements of the target orientation in each dimension.

It should be noted that the above adjusting process may be an adjustment directly performed on the 4D ultrasound image. For example, after the difference between the current orientation and the target orientation of the at least one anatomical feature of interest is calculated by the processor, the 4D ultrasound image obtained by the processing is adjusted, such as deflected, so that the at least one anatomical feature of interest reaches the target orientation. In addition, in the above embodiments of the present application, each image frame of the 4D ultrasound image will be adjusted according to the above target orientation, thereby ensuring that the above at least one structure of interest has the target orientation in the entire movie sequence of the 4D ultrasound image. In some other examples, the above adjusting process may consist of adjusting the 4D ultrasound data. For example, for example, after the difference between the current orientation and the target orientation of the at least one anatomical feature of interest is calculated by the processor, the 4D ultrasound data may be first adjusted, such as deflected, and after the adjustment of the 4D ultrasound data is completed, the 4D ultrasound data is then processed, so as to obtain the adjusted 4D ultrasound image. It can be understood that in the 4D ultrasound image generated by means of the above adjustment, the structure of interest in each volumetric image frame will be maintained at the target orientation.

By means of the above embodiments of the present application, the feature of interest in the volumetric images will always be maintained at the target orientation, thereby avoiding the cumbersome workflow resulting from manually moving the probe or operating adjustment functions of the ultrasound imaging system, and high quality ultrasound images can be obtained easily even if the operation is performed by a novice. Furthermore, more importantly, the present application enables each volumetric image frame in the 4D ultrasound image to be adjusted, so that the target orientation of the anatomical feature of interest can be maintained even in the case of a moving ultrasound probe or a moving tissue to be imaged (e.g., the fetus or the heart), which is difficult to achieve by manual operation.

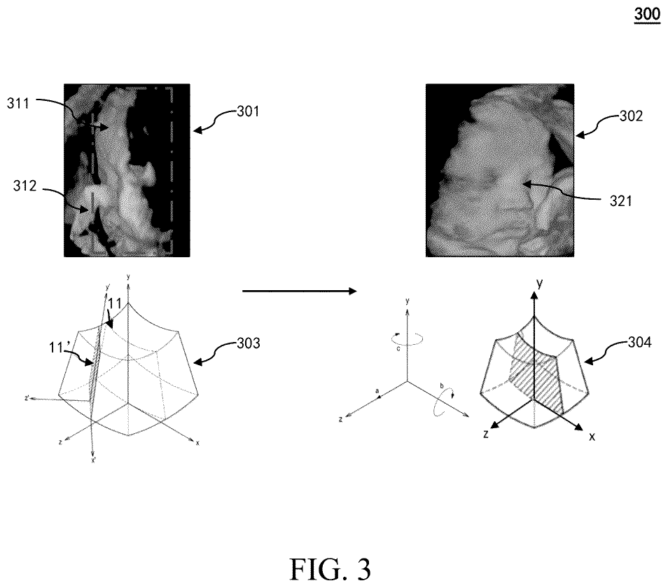

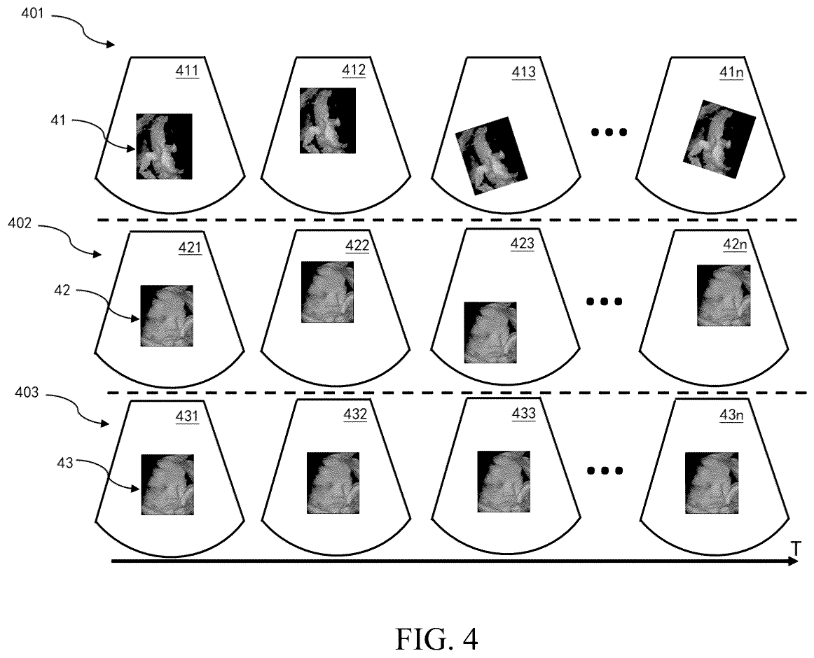

The adjustment of the 4D ultrasound image in the present application is explained in more detail below by means of drawings. Reference is made to FIGS. 3 and 4. FIG. 3 illustrates a schematic diagram 300 showing how adjustments are made to a volumetric image in some embodiments of the present application, and FIG. 4 illustrates a schematic diagram 400 showing how orientation adjustments are made in a plurality of volumetric images in some embodiments of the present application.

First, with reference to FIG. 3, an anatomical feature of interest 311 (using the face of a fetus as an example) is identified in an unadjusted volume 303, and the plane in which the feature is located is represented by 11′. It can be understood that the volume may represent volumetric data that has not been processed, and may represent a volumetric image obtained after the processing. At such time, an image 301 of the anatomical feature of interest after imaging is as shown in FIG. 3. Since the orientation is lateral with respect to an observer, much information about the face of the fetus will be obscured, and the ultrasound scanning technician will need to adjust the volumetric image or move the probe to ultimately obtain an satisfactory image of the face of the fetus.

In the embodiments of the present application, according to the type of the identified anatomical feature of interest 311 as shown, the processor can automatically determine a target orientation thereof (plane 11 in the figure) and calculate the difference between the current orientation (plane 11′) and the target orientation, such that the volume is adjusted, such as deflected. As shown in FIG. 3, an anatomical structure of interest 321 having the target orientation can be presented clearly in an adjusted volume 304. At such time, the face of the fetus can be completely displayed. A variety of adjustment methods may be used. For example, the volume may be rotated by a suitable angle in X-axis, Y-axis, and Z-axis directions as desired, so as to obtain the desired target orientation.

It can be understood that the volume shown in FIG. 3 is volumetric data or a volumetric image. In the present application, the 4D data or 4D image is understood as a sequence of the volumetric data or volumetric images described above in the time dimension. The adjustment to the orientation of the anatomical feature of interest in the 4D image may be understood as an adjustment made to each volumetric image frame, and details thereof will not be described herein again.

Furthermore, in addition to adjusting the 4D ultrasound image to ensure that the anatomical feature of interest is maintained at the target orientation in the image, some embodiments of the present application further include other adjustments made to the ultrasound image. In one example, the adjustment may further include at least partially removing anatomical features that obscure the anatomical feature of interest in a viewing direction. With reference to FIG. 3, in the unadjusted volumetric image 301, the front of the anatomical feature of interest 311 (e.g., the face of the fetus) is obscured by another anatomical feature 312. In this case, even if the orientation is adjusted, it is difficult to meet requirements, because the ultrasound examination physician cannot clearly and completely observe the entire fetal face. In some embodiments of the present application, an improvement is made, that is, the adjustment of the ultrasound image further includes removing the above anatomical feature obstructing the view in a viewing direction. In the above adjusted ultrasound image 302, the anatomical feature of interest 321 can be completely exposed in the viewing direction (or the direction of observation of the observer), thereby facilitating observation. It can be understood that the above-described means of removing may be implemented in the volumetric data based on the identification of the anatomical feature of interest. After identifying the anatomical feature of interest, the processor may remove data of other anatomical features in front of data of the anatomical feature of interest at this time, thereby exposing the anatomical feature of interest. In addition, since the original volumetric data of the anatomical feature of interest is not lost or distorted due to the removal, the volumetric image that has undergone the removal processing can still accurately reflect the actual appearance of the anatomical feature of interest.

In some other embodiments, the adjustment of the 4D ultrasound image may further include maintaining the at least one anatomical structure of interest always at a fixed location in the plurality of image frames. A detailed description is provided with reference to FIG. 4. FIG. 4 shows changes in different volumetric image frames in the time dimension (the horizontal coordinates t).

First, reference is made to an image frame sequence 401, which includes a plurality of volumetric image frames, including 411 to 41n. These volumetric images 411 to 41n have not undergone any adjustments, including orientation and position, i.e., the images represent the current orientation of an anatomical feature of interest 41. Using the face of a fetus as an example, in the image frame sequence 401, the face of the fetus is always oriented laterally, and the complete facial information cannot be displayed. Furthermore, due to the movement of the probe or the activity of the fetus, the position of the anatomical feature of interest 41 in the field of view is also constantly changing. It will be difficult for the ultrasound examination physician to complete valid information in the frame sequence 401.

In some embodiments according to the present application, the above image frame sequence 401 is adjusted to ensure that the anatomical feature of interest 41 is maintained at a target orientation in the frame sequence. The adjustment result is as shown by an image frame sequence 402, which includes a plurality of volumetric images 421 to 42n. In comparison to the frame sequence 401, the orientation of an anatomical feature of interest 42 in the frame sequence 402 has been adjusted so that said feature of interest is controlled to be maintained at the target orientation. At such time, key features in the anatomical feature of interest 42 can be easily observed, thereby facilitating ultrasonic examination by the physician. However, as noted above, the probe or the fetus may move in the examination process. As such, even if the anatomical feature of interest 42 is adjusted to be at the target orientation, the anatomical structure of interest 42 may be translated within the plane of the target orientation, as shown in the sequence 402. That is, in the entire 4D ultrasound image, shaking will occur for the anatomical feature of interest 42 at different positions. When the frame rate is too fast, the shaking will be more pronounced.

In some other embodiments according to the present application, the adjustment may further include maintaining the at least one anatomical structure of interest always at a fixed location in the plurality of image frames. Reference is made to a frame sequence 403, which contains a plurality of volumetric image frames 431 to 43n. These volumetric image frames 431 to 43n undergo orientation adjustment to ensure that the image frames all have the target orientation. Moreover, the positions of the images frames on the image are adjusted to ensure that the image frames are all positioned at the center of the image. As shown by the frame sequence 403, an anatomical feature of interest 43 can remain stable in all of the plurality of volumetric image frames 431 to 43n. That is, in the 4D ultrasound image, even if other anatomical features in the volumetric image are moving, shaking does not occur for the anatomical feature of interest 43, thereby further facilitating the observation by the physician.

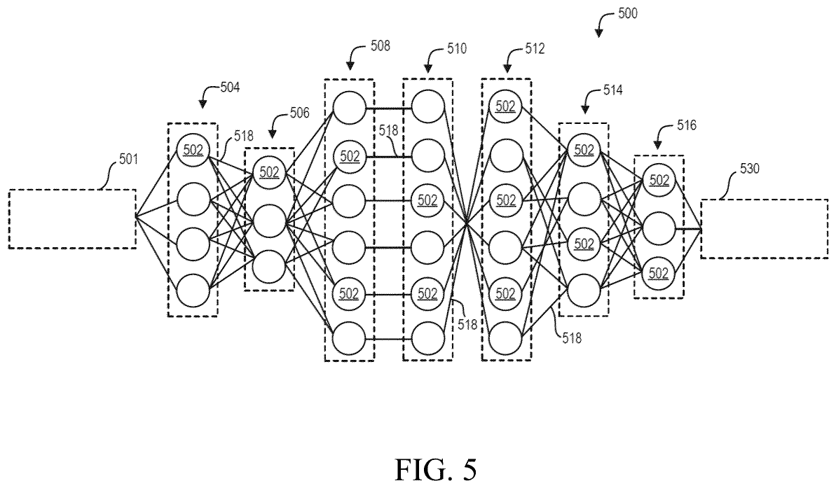

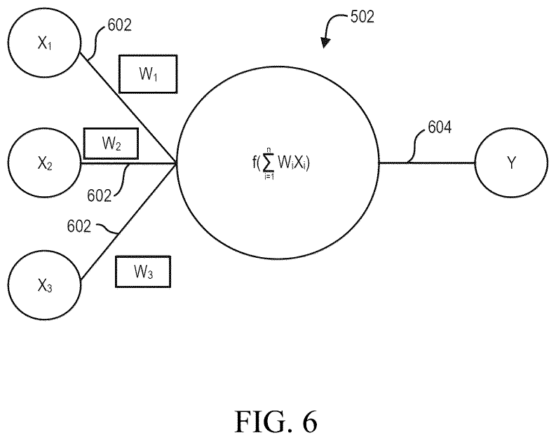

As described above, the identification of the anatomical feature of interest may be implemented using an image recognition algorithm stored, for example, in the memory 106. There may be a variety of types of image recognition algorithm. In one example, the identification may be implemented by means of pattern recognition. In another example, the identification may also be implemented by means of artificial intelligence. An exemplary description is provided below. With reference to FIGS. 5 and 6, depicted is an exemplary neural network for identifying and classifying one or more anatomical features from 4D ultrasound data of a tissue to be imaged, e.g., a fetus. In some examples, prior to identifying the one or more anatomical features from the 4D ultrasound data input, the neural network may be trained using a training set of 4D ultrasound data depicting other fetuses. In an example in which the one or more anatomical features include one or more facial features, the neural network may be considered to be a facial recognition algorithm.

FIG. 5 depicts a schematic diagram of a neural network 500 having one or more nodes/neurons 502. In some embodiments, the one or more nodes/neurons may be disposed in one or more layers 504, 506, 508, 510, 512, 514, and 516. The neural network 500 may be a deep neural network. As used herein with respect to the neurons, the term “layer” refers to a set of analog neurons having inputs and/or outputs connected to other sets of analog neurons in a similar manner. So, as shown in FIG. 5, the neurons 502 may be connected to each other via one or more connections 518, such that data may be propagated from an input layer 504, through one or more intermediate layers 506, 508, 510, 512, and 514, to an output layer 516.

FIG. 6 shows input and output connections of a neuron according to an exemplary embodiment. As shown in FIG. 6, connections (e.g., 518) of a single neuron 502 may include one or more input connections 602 and one or more output connections 604. Each input connection 602 of the neuron 502 may be an output connection of a leading neuron, and each output connection 604 of the neuron 502 may be an input connection of one or more subsequent neurons. While FIG. 6 depicts the neuron 502 as having a single output connection 604, it should be understood that the neuron may have multiple output connections that send/transmit/pass the same value. In some embodiments, the neuron 502 may be a data construct (e.g., a structure, an instantiated class object, a matrix, etc.), and the input connections 602 may be received by the neuron 502 as weighted values (e.g., floating points or integer values). For example, as further shown in FIG. 6, input connections X1, X2, and X3 may be weighted using weights W1, W2, and W3, respectively, summed, and sent/transmitted/passed as an output connection Y. As will be understood, the processing by a single neuron 502 may generally be represented by the following equation:

$$Y = f ( {\sum }_{i = 1}^{n} {W}_{i} {X}_{i} )$$

where n is the total number of the input connections 602 to the neuron 502. In one embodiment, the value of Y may be based at least in part on whether the sum of WiXis exceeds a threshold. For example, if the sum of the weighted inputs does not exceed a desired threshold, Y may have the value of zero (0).

As will be further understood from FIGS. 5 and 6, the input connections 602 of the neuron 502 in the input layer 504 may be mapped to an input 501, and the output connection 604 of the neuron 502 in the output layer 516 may be mapped to an output 530. As used herein, “mapping” a given input connection 602 to the input 501 refers to the way in which the input 501 affects/indicates the value of the input connection 602. Similarly, as used herein, “mapping” a given output connection 604 to the output 530 refers to the way in which the value of the output connection 604 affects/indicates the output 530.

Hence, in some embodiments, the acquired/obtained input 501 is passed/fed to the input layer 504 of the neural network 500 and propagated through the layers 504, 506, 508, 510, 512, 514, and 516, such that the mapped output connection 604 of the output layer 516 generates/corresponds to the output 530. As shown, the input 501 may include 4D ultrasound data, and the 4D ultrasound data has comprehensive imaging information, and shows one or more anatomical features (such as one or more facial features, e.g., a nose, a mouth, eyes, cars, etc.) that can be identified by the neural network 500. Furthermore, the output 530 may include the location and classification of one or more identified anatomical features. For example, the neural network 500 may identify an anatomical feature depicted by a render, generate coordinates indicating the location of the anatomical feature (e.g., at the center, or on the perimeter), and classify the anatomical feature (e.g., the nose) based on the identified visual characteristics. Accordingly, the type of the anatomical feature and an orientation thereof before the adjustment can be obtained. In an example in which the neural network 500 is a facial recognition algorithm, the output 530 may specifically include one or more facial features.

The neural network 500 may be trained using a plurality of training data sets. Each training data set may include 4D ultrasound data depicting one or more anatomical features of other fetuses. Thus, the neural network 500 can learn relative positions and shapes of the one or more anatomical features depicted in the 4D ultrasound data. In this way, the neural network 500 may utilize the plurality of training data sets to map the generated 4D ultrasound data (e.g., inputs) to one or more anatomical features (e.g., outputs). Machine learning or deep learning (e.g., due to a recognizable trend in the arrangement, size, etc., of the anatomical feature) may cause changes in weights (e.g., W1, W2, and/or W3), changes in input/output connections, or other adjustments to the neural network 500. Furthermore, as additional training data sets are employed, as a response, various parameters of the neural network 500 may be adjusted by the machine learning continuously. As such, the sensitivity of the neural network 500 can be periodically increased, resulting in higher anatomical feature identification precision.

It can be understood that the above is an exemplary description of a neural network for identifying an anatomical feature of interest and a current orientation thereof, and the embodiments of the present application are not limited thereto.

As described above, when using a means such as the neural network to identify the 4D ultrasound data, a plurality of anatomical structures of interest may be identified therein. In one example, the target orientation is an orientation at which the plurality of anatomical structures of interest described above can be shown simultaneously. However, in another example, it is possible to carry out orientation for each of the plurality of anatomical structures of interest described above. Various embodiments and advantages thereof will be described in detail below.

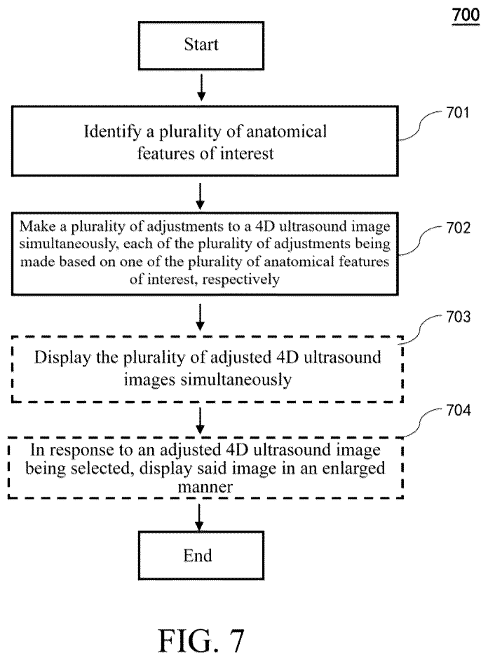

First, with reference to FIG. 7, provided is a flowchart 700 showing how a 4D ultrasound image including a plurality of anatomical features of interest is adjusted in some embodiments of the present application.

In step 701, a plurality of anatomical features of interest are identified. The means of identification may be as described in any of the embodiments described above. For example, when the neural network is trained, different anatomical features of interest may be separately used as the output, and correspondingly, when the neural network is used, a plurality of anatomical features of interest may be identified simultaneously, including orientations thereof.

In step 702, a plurality of adjustments are made to the 4D ultrasound image simultaneously, each of the plurality of adjustments being made based on one of the plurality of anatomical features of interest, respectively. The process may be implemented by the processor. In embodiments of the present application, after identifying the plurality of anatomical features of interest, the processor may make the plurality of adjustments to the 4D ultrasound image simultaneously. That is, during real-time ultrasonic scanning, the scanning operator does not need to perform a plurality of scans, and a plurality of adjustments of different anatomical features of interest can be achieved in only one scan process. As noted in the above embodiments herein, the adjustments can be made to the 4D ultrasound image or the 4D ultrasound data, and details thereof will not be described herein again. By means of step 702, each of the identified anatomical features of interest undergoes targeted adjustment, that is, a 4D ultrasound image having the target orientation of this anatomical feature of interest is obtained. In this way, different anatomical features of interest correspond to different adjusted 4D ultrasound images.

In addition to the different target orientations configured according to the different features of interest, in one embodiment, different adjustment parameters can be further configured for the multiple sets of adjustments according to differences between the multiple anatomical features of interest. Such a configuration manner enables more precise adjustments to be made to the 4D ultrasound image of a particular anatomical feature of interest. The different adjustment parameters include at least one of different target orientations, different transparencies, different lighting directions, and different lighting colors.

For example, different anatomical features of interest may have different target orientations. For example, for the face of a fetus, a preferred target orientation may be the plane in which the face is located tilted by 10-20 degrees. In this way, on one hand, the physician can observe the entire facial structure, and on the other hand, a slight tilt can facilitate determining whether the facial structure has a slight defect. For another example, for a hand of a fetus, since the hand is moving constantly, at such time, the target orientation will vary depending on the current location of the hand, and a preferred target orientation will be a viewing direction in which all fingers can be clearly seen simultaneously. Similarly, the transparency, lighting direction, and lighting color of a 4D render can affect the appearances of different anatomical features of interest, and may also affect the observation of the tissue to be imaged. Imparting the above imaging parameters with different values for different anatomical features of interest will facilitate observation by the pregnant woman and the physician.

By means of the above configuration, when there are a plurality of anatomical features of interest, the processor can perform different types of processes on the 4D ultrasound data respectively within the same period of time, without needing to perform multiple scans repeatedly. Each of the plurality of processes is respectively for an anatomical feature of interest. When this solution is applied to real-time scanning, the scanning operator only needs to perform one scan to obtain a plurality of 4D ultrasound images for different anatomical features of interest, which not only has high imaging effects, but also greatly saves time.

In one example, the above plurality of 4D ultrasound images that have been processed may be stored. It is recognized by the inventor that if each processed 4D ultrasound image is stored, a large amount of space of the memory may be occupied. Improvements are provided in an embodiment of the present application. In one embodiment, an adjustment record for each image frame in a 4D ultrasound image may be stored. That is, after an original 4D ultrasound image is adjusted, the processor may save the adjustment parameters instead of the adjusted 4D ultrasound image. For example, the processor may save the angle of coordinate transformation (orientation adjustment), transparency, lighting direction, lighting color, and the like. The adjustment information occupies only a small space. By such a configuration manner, whether one or a plurality of anatomical features of interest are identified, the processor can perform complete and fast recording after adjusting the 4D ultrasound image.

Furthermore, optionally, in step 703, the plurality of adjusted 4D ultrasound images are displayed simultaneously. The process may be implemented by the processor. On the basis that the plurality of anatomical features of interest are identified and the orientations and/or other parameters thereof are adjusted, the processor can obtain a plurality of 4D images for different anatomical features of interest, respectively. At such time, the images are displayed on the display, thereby enabling the ultrasound scanning physician to easily and promptly understand the anatomical features that have been scanned, so as to until the next step of the scanning work.

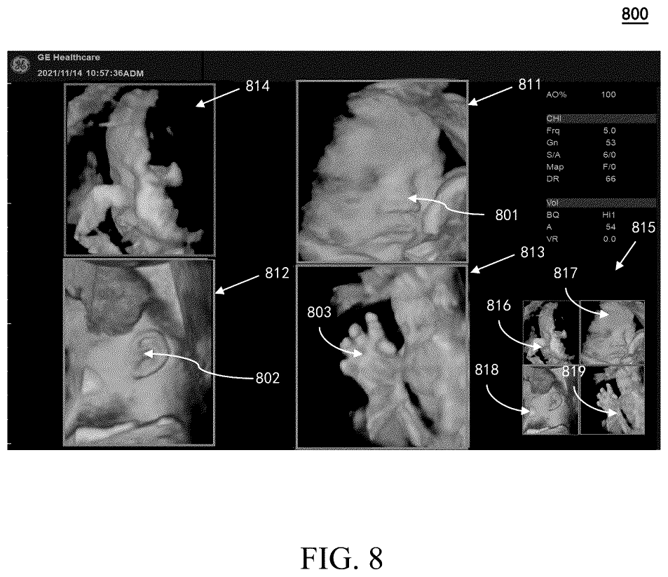

In the following, the simultaneous display will be explained in detail by means of illustrations. With comprehensive reference to FIG. 8, a schematic diagram 800 of simultaneous display of a plurality of adjusted 4D ultrasound images in some embodiments of the present application is shown. In FIG. 8, 4D ultrasound data is obtained using the head of a fetus as the tissue to be scanned, but it can be understood that the tissue to be scanned during actual operation may be arbitrary. Further, the processor identifies, from the 4D ultrasound data of the head of the fetus, three different anatomical features of interest, which are respectively the face of the fetus 801, an ear of the fetus 802, a hand of the fetus 803. As noted in the above embodiments of the present application, the processor may adjust the orientation and other parameters for these different features of interest, respectively, so as to facilitate observations of the different features of interest. The above three anatomical features of interest are displayed simultaneously, and ultrasound images 811 to 813 in which the three anatomical features of interest are located are configured with different orientations and other imaging parameters, respectively, thereby facilitating observation.

In one example, in addition to the anatomical features of interest being displayed, an unprocessed 4D ultrasound image 814 may also be displayed. The unprocessed 4D ultrasound image 814 may be adjusted by the scanning operator autonomously, to avoid all 4D ultrasound images processed by the processor failing to meet clinical requirements. In another example, the above displaying may further include displaying ultrasound image thumbnails. With reference to a thumbnail region 815 in FIG. 8, an unprocessed 4D ultrasound image thumbnail 816 and multiple sets of adjusted 4D ultrasound image thumbnails 817 to 819 are illustrated, respectively.

Optionally, in step 704, in response to an adjusted 4D ultrasound image being selected, said image is displayed in an enlarged manner. The means of selection may be arbitrary. For example, in some embodiments, the selection may be made by means of any user input device, such as a keyboard, mouse, touchscreen, trackball, etc. After receiving the above instruction, the processor may display the selected 4D ultrasound image in an enlarged manner. There may be a variety of enlarged-display methods. In one example, the ultrasound image displayed in an enlarged manner may be suspended on other non-enlarged ultrasound images. In another example, said image may be configured to be displayed alone on the display.

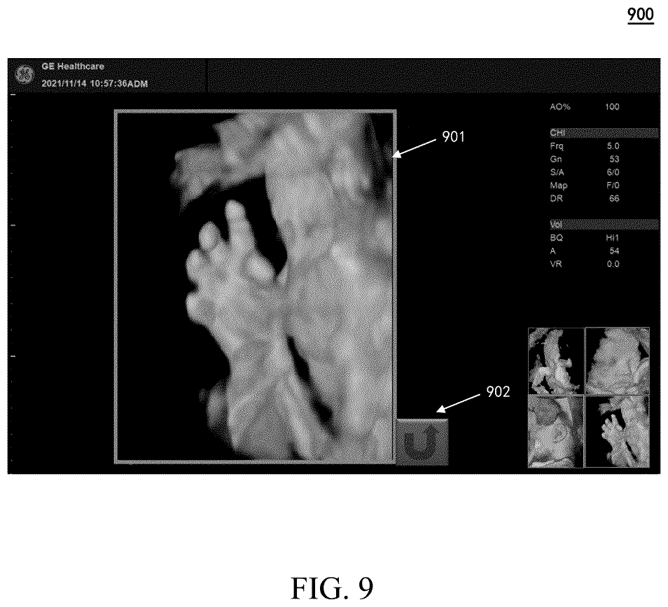

With reference to FIG. 9, a schematic diagram 900 of a 4D ultrasound image displayed in an enlarged manner is shown. As described above, the means of selection may be arbitrary. After being selected, a 4D ultrasound image 901 is displayed in an enlarged manner on the display, thereby facilitating the observation of details by the scanning operator.

Furthermore, in one example, the 4D ultrasound image displayed in an enlarged manner is further configured to be provided with a return key 902. By operating the return key, it is possible to return to the screen in the previous step, enabling the scanning operator to easily reselect other 4D ultrasound images that need to undergo the operation. By such a configuration manner, whether enlarging a certain 4D ultrasound image or returning to a previous operation, both are very fast.

Some embodiments of the present invention further provide a system for adjusting an orientation of a 4D ultrasound image. The system may be the ultrasound imaging system as shown in FIG. 1, or may be any other system. The system includes: a probe, configured to acquire ultrasound data; a processor, configured to perform the method in any of the above embodiments; and a display, configured to receive a signal from the processor and perform a display operation.

Some embodiments of the present invention further provide a non-transitory computer-readable medium storing a computer program, wherein the computer program has at least one code segment, and the at least one code segment is executable by a machine so that the machine performs steps of the method in any of the embodiments described above.

Correspondingly, the present disclosure may be implemented as hardware, software, or a combination of hardware and software. The present disclosure may be implemented in at least one computer system by using a centralized means or in a distributed means, different elements in the distributed means being distributed on a number of interconnected computer systems. Any type of computer system or other device suitable for implementing the methods described herein is considered to be appropriate.

The various embodiments may also be embedded in a computer program product, which includes all features capable of implementing the methods described herein, and the computer program product is capable of executing these methods when loaded into a computer system. The computer program in this context means any expression in any language, code, or symbol of an instruction set intended to enable a system having information processing capabilities to execute a specific function directly or after any or both of a) conversion into another language, code, or symbol; and b) duplication in a different material form.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, and changes can also be made to the present invention and should be included in the scope of protection of the present invention as long as these changes do not depart from the spirit of the present invention.

1. A system, comprising:
a probe, configured to receive 4D ultrasound data about a tissue to be imaged;
a memory storing instructions;
a processor, configured to execute the instructions to:
acquire 4D ultrasound data obtained from a tissue;
process the 4D ultrasound data to generate a 4D ultrasound image, the 4D ultrasound image comprising a plurality of image frames;
identify at least one anatomical feature of interest and a current orientation thereof; and
adjust the 4D ultrasound image, such that the at least one anatomical feature of interest is maintained at a target orientation in the plurality of image frames; and
a display, configured to receive a signal from the processor and performing a display operation.

2. The system according to claim 1, wherein the processor is configured to execute the instructions to identify at least one anatomical feature of interest by identifying the at least one anatomical feature of interest in the 4D ultrasound data.

3. The system according to claim 1, wherein the processor is configured to execute the instructions to adjust the 4D ultrasound image by:
at least partially removing an anatomical feature obscuring the anatomical feature of interest in a viewing direction; and/or
maintaining the at least one anatomical feature of interest always at a fixed location in the plurality of image frames.

4. The system according to claim 1, wherein the at least one anatomical feature of interest comprises a plurality of anatomical features of interest, and the processor is configured to execute the instructions to adjust the 4D ultrasound image by:
making a plurality of adjustments to the 4D ultrasound image simultaneously, each of the plurality of adjustments being made based on one of the plurality of anatomical features of interest, respectively.

5. The system according to claim 4, wherein
the processor is configured to execute the instructions to adjust the 4D ultrasound image by: configuring different adjustment parameters for the plurality of sets of adjustments according to differences between the plurality of anatomical features of interest; and
the different adjustment parameters comprise at least one of different target orientations, different transparencies, different lighting directions, and different lighting colors.

6. The system according to claim 4, wherein the processor is further configured to execute the instructions to:
display the plurality of adjusted 4D ultrasound images simultaneously.

7. A method, comprising:
acquiring 4D ultrasound data obtained from a tissue;
processing the 4D ultrasound data to generate a 4D ultrasound image, the 4D ultrasound image comprising a plurality of image frames;
identifying at least one anatomical feature of interest and a current orientation thereof; and
adjusting the 4D ultrasound image, such that the at least one anatomical feature of interest is maintained at a target orientation in the plurality of image frames.

8. The method according to claim 7, wherein
the 4D ultrasound data comes from at least one of a real-time ultrasonic scan and data in a memory.

9. The method according to claim 7, wherein
the identifying at least one anatomical feature of interest comprises identifying the at least one anatomical feature of interest in the 4D ultrasound data.

10. The method according to claim 7, wherein the adjusting the 4D ultrasound image further comprises:
at least partially removing an anatomical feature obscuring the anatomical feature of interest in a viewing direction.

11. The method according to claim 7, wherein the adjusting the 4D ultrasound image further comprises:
maintaining the at least one anatomical feature of interest always at a fixed location in the plurality of image frames.

12. The method according to claim 7, further comprising:
storing an adjustment record for each image frame in the 4D ultrasound image.

13. The method according to claim 7, wherein the at least one anatomical feature of interest comprises a plurality of anatomical features of interest, and the adjusting the 4D ultrasound image further comprises:
making a plurality of adjustments to the 4D ultrasound image simultaneously, each of the plurality of adjustments being made based on one of the plurality of anatomical features of interest, respectively.

14. The method according to claim 13, wherein the adjusting the 4D ultrasound image further comprises:
configuring different adjustment parameters for the plurality of sets of adjustments according to differences between the plurality of anatomical features of interest.

15. The method according to claim 14, wherein the different adjustment parameters comprise at least one of different target orientations, different transparencies, different lighting directions, and different lighting colors.

16. The method according to claim 13, further comprising:
displaying the plurality of adjusted 4D ultrasound images simultaneously.

17. The method according to claim 16, further comprising:
in response to an adjusted 4D ultrasound image being selected, displaying said image in an enlarged manner.

18. A non-transitory computer-readable medium, storing a computer program, the computer program having at least one code segment, and the at least one code segment being executable by a machine to cause the machine to:
acquire 4D ultrasound data obtained from a tissue;
process the 4D ultrasound data to generate a 4D ultrasound image, the 4D ultrasound image comprising a plurality of image frames;
identify at least one anatomical feature of interest and a current orientation thereof; and
adjust the 4D ultrasound image, such that the at least one anatomical feature of interest is maintained at a target orientation in the plurality of image frames.

19. The non-transitory computer-readable medium according to claim 18, wherein the adjusting the 4D ultrasound image further comprises:
at least partially removing an anatomical feature obscuring the anatomical feature of interest in a viewing direction; and/or
maintaining the at least one anatomical feature of interest always at a fixed location in the plurality of image frames.

20. The non-transitory computer-readable medium according to claim 18, wherein the at least one anatomical feature of interest comprises a plurality of anatomical features of interest, and the adjusting the 4D ultrasound image further comprises:
making a plurality of adjustments to the 4D ultrasound image simultaneously, each of the plurality of adjustments being made based on one of the plurality of anatomical features of interest, respectively.
